(19) United States
(12) Patent Application Publication
(10) Pub. No.: US 2010/0049252 A1
(43) Pub. Date: Feb. 25, 2010

(54) Transverse Connector Device for Extending an Existing Spinal Fixation System

(75) Inventors: Hugh F. Smisson, III, Macon, GA (US); Michael A. Cowan, Charlotte, NC (US)

Correspondence Address: SUTHERLAND ASBILL & BRENNAN LLP, 999 PEACHTREE STREET, N.E., ATLANTA, GA, 30309, US

(73) Assignee: SOUTHERN SPINE, LLC, Macon, GA (US)

(21) Appl. No.: 12/545,661

(22) Filed: Aug. 21, 2009

Related U.S. Application Data
(60) Provisional application No. 61/090,731, filed on Aug. 21, 2008.

(51) Int. Cl. A61B 17/70; A61B 17/88

(52) U.S. Cl. 606/250; 606/264; 606/279

(57) ABSTRACT

An extension rod device is provided for extending an existing spinal fixation system. The device can engage with implanted spinal fixation elements and house extension spinal rods that expand the fixation system to adjacent vertebrae. According to various embodiments, an extension rod device may include one or more extension spinal rods attachable to a transverse connector that is secured between an existing, implanted spinal fixation system. The extension rod device may attach to an existing transverse connector, already implanted with the existing spinal fixation system, or the extension rod device may include a new transverse connector, also for securing to the existing spinal fixation system.

### CROSS REFERENCES TO RELATED APPLICATIONS

This application claims the priority benefit of Provisional Patent Application Ser. No. 61/090,731 filed on Aug. 21, 2008, which is hereby incorporated by reference in its entirety as if set forth fully herein.

### FIELD OF THE INVENTION

The present invention relates generally to devices for spinal fixation, and more particularly to devices for extending an existing spinal fixation system to adjacent vertebrae.

### BACKGROUND OF THE INVENTION

Implantable spinal fixation systems are used to surgically treat a variety of spinal disorders. Such systems typically consist of two spinal fixation elements for fixing adjacent vertebrae, such as rods or plates, which are secured to opposite sides of the spinal column by attachment elements, such as screws or hooks. The spinal fixation elements can be connected by a transverse connector which extends substantially horizontally between the spinal fixation elements and across a fixated vertebra. Transverse connectors are known to provide increased strength and stability to spinal fixation systems.

A fixation system is surgically implanted at the disordered portion of the spine. Spinal fixation systems of various lengths are available, and an appropriate length can be chosen to cover any number of vertebrae that require fixation. Typically only one or two vertebrae require fixation during the initial procedure.

However, it is not uncommon for vertebrae adjacent to the fixated vertebrae to require fixation at some point after the initial fixation procedure. The adjacent vertebrae may degenerate, in part, due to the load of the initially implanted fixation system. Fixation may also be required to treat fractures, herniations, and other disorders of the adjacent vertebrae. Thus, it is often necessary to add a fixation system to vertebrae adjacent to the vertebrae on which a fixation system is initially implanted.

Most, if not all, presently available spinal fixation systems do not allow an existing fixation system to be extended through the addition of fixation elements, unless the existing fixation system is specifically designed to receive extensions. Instead, to add a fixation system to adjacent vertebrae, most existing systems must be removed and a new, longer system implanted. Removal of the existing system and implantation of an entire new system has several disadvantages, including increasing the length of the surgical procedure and the risk associated therewith. Also, removal and re-implantation of the fixation elements requires removing the first set of pedicle screws from the vertebrae, which can result in the removal of bone material from the vertebrae and cause extra wear and tear. Thus, it is desirable to have a device which allows the extension of an existing spinal fixation system to adjacent vertebrae without removal of the existing system.

Therefore, there remains a need in the art for an improved device which allows the extension of an existing spinal fixation system to adjacent vertebrae.

### SUMMARY OF THE INVENTION

New devices and methods are provided for extending an existing spinal fixation system. In one aspect, a transverse connector device is provided. In one embodiment, the transverse connector device includes a first lateral end which is adapted to engage a first spinal fixation element, a second lateral end which is adapted to engage a second spinal fixation element, a connecting element between the lateral ends, and at least one housing member which is adapted to receive and secure at least one extension spinal rod.

In one embodiment, each lateral end includes a clamp, which engages the respective spinal fixation element. In another embodiment, each lateral end includes a hook, which engages the respective spinal fixation element. In yet another embodiment, the lateral ends are secured to the existing spinal fixation elements using other attachment elements, such as screws. In one embodiment, the spinal fixation elements are spinal rods. In various embodiments, the connecting element of the transverse connector device can be adjustable along the anteroposterior and/or the superior-inferior axis, and/or can be adjustable in length. In one embodiment, at least one extension spinal rod is included with the transverse connector device, for securing to a vertebra.

According to another aspect, an extension spinal rod for extending an existing spinal fixation system may be provided. The extension spinal rod may have a proximal end adapted to be secured to a transverse connector and a distal end adapted to be secured to a vertebra. The extension spinal rod can be configured to extend from a transverse connector extending between spinal fixation elements at a first vertebral level to at least one vertebra at an adjacent vertebral level.

According to one embodiment, the distal end of the extension spinal rod is secured to a vertebra using a pedicle screw. According to another embodiment, the extension spinal rod can be adjustable along the anteroposterior axis, the mediolateral axis, and/or the superior-inferior axis, and/or can be adjustable in length.

According to one embodiment, at least one housing member is attachable to the proximal end of the extension spinal rod, wherein the housing member is attachable to a transverse connector.

In another aspect, a method is provided for extending an existing spinal fixation system to adjacent vertebrae. In one embodiment, the method includes securing a first lateral end of a transverse connector to a first implanted spinal fixation element, securing a second lateral end of a transverse connector to a second implanted spinal fixation element, securing a proximal end of at least one extension spinal rod to the transverse connector, and securing a distal end of the extension spinal rod to a vertebra.

In yet another aspect, a method is provided for extending an existing spinal fixation system, having an existing transverse connector, to adjacent vertebrae. In one embodiment, the method includes securing a proximal end of at least one extension spinal rod to an existing transverse connector, and securing a distal end of the at least one extension spinal rod to a vertebra.

In one embodiment, securing the proximal end of the extension spinal rod to the existing transverse connector can include attaching at least one housing member to the existing transverse connector, and attaching the proximal end of the extension rod to the housing member.

According to yet another aspect, an extension rod device kit is provided. The extension rod device kit can include at least one extension spinal rod having a proximal end and a distal end, at least one pedicle screw assembly for affixing the distal end of the extension spinal rod to a vertebra, and at least one housing member for receiving the proximal end of the extension rod and for securing to a transverse connector securable between two implanted spinal rods.

In one embodiment, the extension rod device kit can further include the transverse connector.

### BRIEF DESCRIPTION OF THE DRAWINGS

Embodiments of the invention will be better understood and more readily apparent when considered in conjunction with the following detailed description and accompanying drawings which illustrate, by way of example, embodiments of an extension rod device, and in which:

FIG. 1 is a schematic diagram of one example embodiment of an extension rod device.

FIG. 2 is a schematic diagram of one example embodiment of an extension rod device attached to an existing spinal fixation system.

FIG. 3 is a schematic diagram of another example embodiment of an extension rod device attached to an existing spinal fixation system.

FIG. 4 is a schematic diagram of another example embodiment of an extension rod device attached to an existing spinal fixation system.

FIGS. 5A-5E are a series of schematic diagrams of various example embodiments of a lateral end of a transverse connector and securing mechanisms.

FIGS. 6A-6T are a series of schematic diagrams of various example embodiments of a housing member of an extension rod device.

FIGS. 7A and 7B are schematic diagrams of example embodiments for attaching an extension rod device to an existing spinal fixation system.

FIGS. 8A and 8B are schematic diagrams of example embodiments of extension spinal rods.

### DETAILED DESCRIPTION OF THE EXAMPLE EMBODIMENTS

As required, detailed embodiments of the invention are disclosed herein, however, it is to be understood that the disclosed embodiments are merely exemplary of the invention which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure.

As used herein, the terms “comprise,” “comprising,” “comprises,” “include,” “includes,” and any other variations thereof, are intended to be open, non-limiting terms, unless the contrary is specifically indicated.

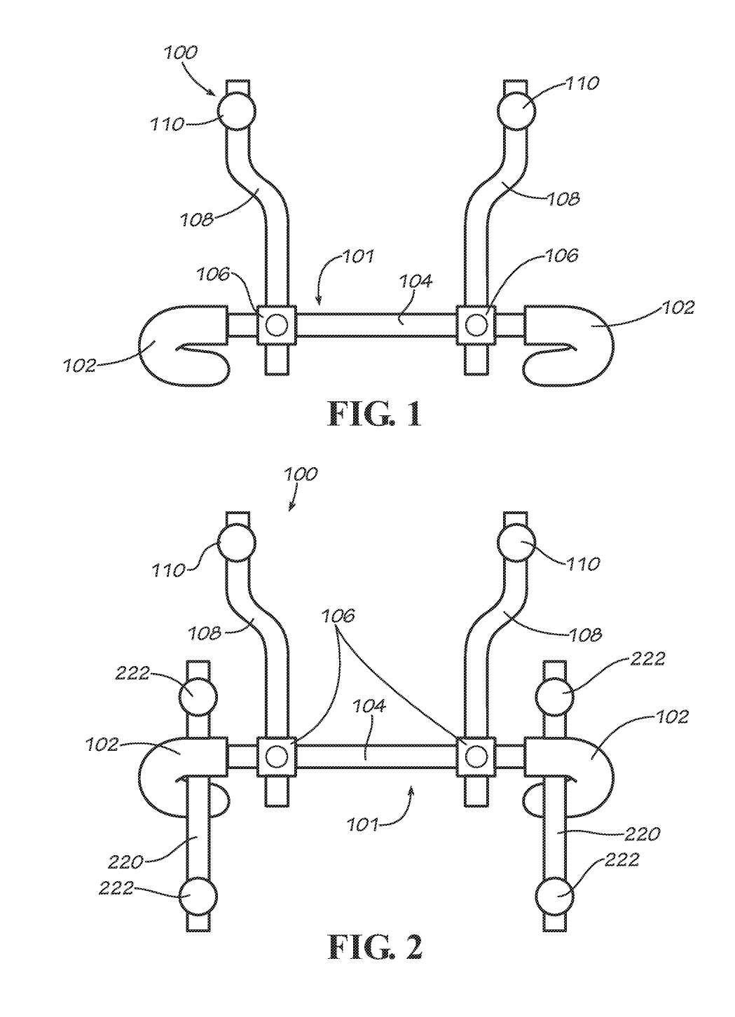

The extension rod device can be further understood with reference to the example, non-limiting embodiments illustrated in FIGS. 1-8. Like numbers refer to like elements throughout the following description.

FIG. 1 shows one embodiment of an extension rod device. The extension rod device 100 of this embodiment includes at least two extension spinal rods 108 (though fewer or more can be included in other embodiments), and a transverse connector 101 having first and second lateral ends 102, and a connecting element 104. The extension rod device 100 also includes at least two housing members 106 for connecting the extension spinal rods 108 to the transverse connector 101. The first and second lateral ends 102 are each adapted to engage one of the implanted spinal fixation elements (further illustrated by FIG. 2), typically rods or plates, to secure the extension rod device 100 to an existing spinal fixation system. The extension rod device 100 is intended to be used with both currently available and future designs of spinal fixation systems. According to one embodiment, previously implanted spinal fixation systems do not have to be specially adapted to receive the extension rod device 100; rather, the extension rod device 100 itself can be adapted to engage the existing fixation system. For example, according to various embodiments, the lateral ends 102 of the transverse connector 101 can be configured to fit a range of available fixation systems. However, in other embodiments, the transverse connector 101 can include lateral ends 102 that are specifically designed to fit a particular fixation system.

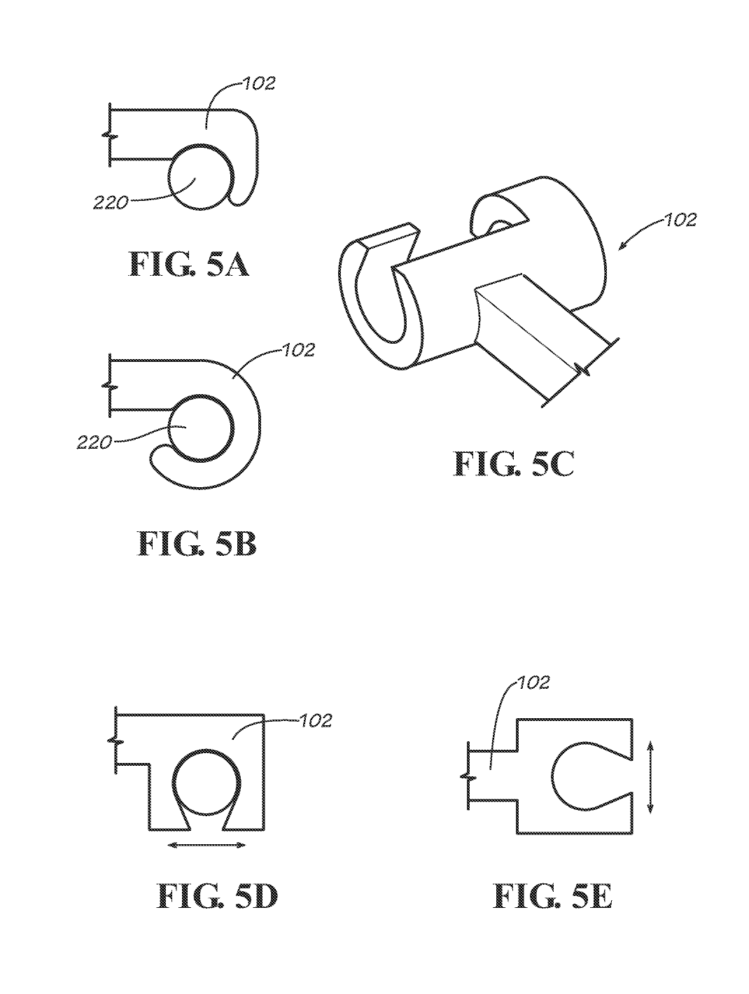

FIG. 2 shows an extension rod device 100 of FIG. 1 attached to an existing implanted spinal fixation system, which consists of two spinal fixation rods 220 secured to a vertebra (not shown) using pedicle screws 222. The lateral ends 102 of the transverse connector 101 can engage the two implanted spinal rods 220 using any securing mechanism or attachment member. Possible securing mechanisms or attachment members include, but are not limited to, hooks, screws, clamps, and the like, example embodiments of which are shown in FIG. 5.

FIGS. 5A and 5B show two single-hook embodiments of securing mechanisms or attachment members at a lateral end of a transverse connector (such as the transverse connector 101, 702 of FIGS. 1-4 or 7). The hook can secure the extension rod device 100 to the spinal rod 220 merely by sliding around it, as shown by the semi-hook embodiment in FIG. 5A, or it can have a snap fit with the spinal rod, as shown by the full-hook embodiment in FIG. 5B. The size and shape of the hook can be chosen according to the spinal rods 220 that the lateral ends are intended to engage. FIG. 5C shows a double-hook embodiment. Like the single-hook embodiments, the size and shape of the hooks can vary, and the hooks can be designed to engage a spinal rod 220 by sliding over it or by having a snap fit. FIGS. 5D and 5E show two different clamp embodiments at the lateral ends 102 of the transverse connector 101. In the embodiment of FIG. 5D, the clamp opens and closes laterally (as indicated by the arrow) and engages the spinal rod 220 from above along the superior-inferior axis. In the embodiment of FIG. 5E, the clamp opens and closes along the superior-inferior axis (as indicated by the arrow) and engages the spinal rod 220 laterally. The clamp can be configured to open and close using a variety of mechanisms, such as, but not limited to, a squeeze-and-release mechanism, a ratchet mechanism, or a screw mechanism.

It is appreciated that the transverse connector 101 can be secured to the spinal rods 220 using any other number of attachment elements not described herein, such as screws. In addition, according to various embodiments, the lateral ends 102 of the transverse connector 101 can be designed for all or some of the edges to be smooth, to minimize potential irritation in the patient's body from contact therewith.

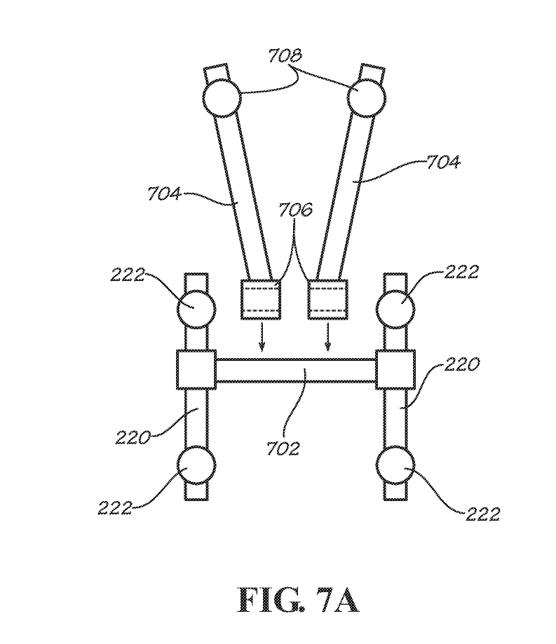

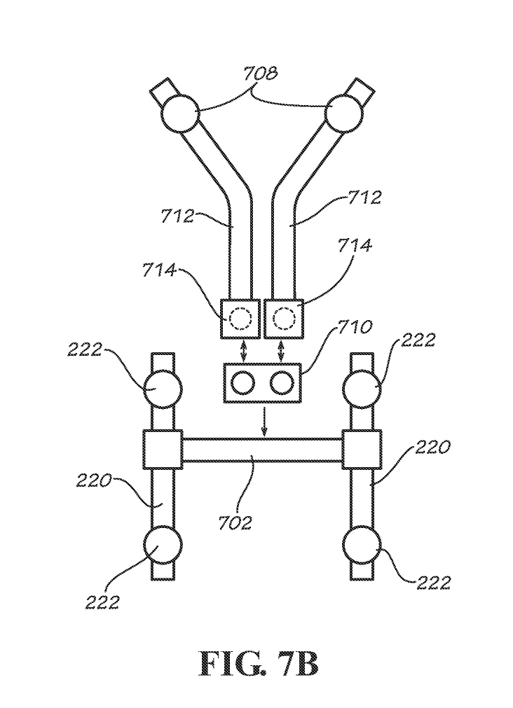

Moreover, as further described below, according to various other embodiments, the extension rod device may not include a transverse connector, but may be configured to secure to an existing implanted transverse connector, such as those example embodiments illustrated in FIGS. 7A and 7B. In these embodiments, the transverse connector likely will already be secured to the implanted spinal rods 220 by any number of means.

With continued reference to FIGS. 1 and 2, the connecting element 104 connects the two lateral ends 102 of the transverse connector 101. The lateral ends 102 and the connecting element 104 can be made as a single piece, or they can be separate pieces, with each lateral end 102 attached to the connecting element 104. The size and shape of the connecting element 104 can vary. The width and depth can be chosen to provide the necessary strength for stabilizing the existing fixation system. The edges of the connecting element 104 can be rounded to minimize potential irritation in the patient's body. The length of the connecting element 104 may be adjustable to account for variations in the separation distance of the spinal rods 220 in the existing spinal fixation system. For example, according to one embodiment, the ends of the connecting element 104 can be slidably attached to each lateral end 102, such that the length between the lateral ends 102 can be adjusted by sliding the connecting element 104 further in or out of the lateral ends 102. In another embodiment, the connecting element 104 itself comprises a sliding adjustment mechanism. In either embodiment, the sliding adjustment mechanism may lock in place at the desired length. The locking may occur by any mechanism, such as, but not limited to, corresponding holes and protrusions that fit together, clamping mechanisms, or a screw mechanism (e.g., one or more set screws). Adjustment of the length can be performed before a surgical procedure, during a surgical procedure, or both.

It is not uncommon for implanted spinal fixation rods 220 to lack level orientation. Advantageously, the connecting element 104 of the transverse connector 101 may be adjustable along the superior-inferior axis to account for differences in the superior-inferior locations of the implanted spinal rods to which the extension rod device is being secured. The adjustment may be achieved by any mechanism. In one embodiment, the connecting element 104 contains a ball joint or other pivot mechanism at some point along its length, which allows one or both sides of the connecting element 104 to be moved relative to the pivot in the anterior and/or posterior direction. This may be performed if the implanted spinal fixation rods 220 lack parallel orientation, for example. In another embodiment, all or a portion of the transverse connector 101 may be bendable or otherwise moldable to allow adjustment in the anterior and/or posterior direction. Adjustment could occur before or during the surgical procedure, including after the lateral ends 102 have engaged the existing fixation system. In embodiments in which the transverse connector 101 is added as a component of the extension rod device, the transverse connector 101 may serve to stabilize the existing system while also allowing its extension to adjacent vertebrae via the extension spinal rods 108.

Each extension spinal rod 108 of the extension rod device 100 has a proximal end, which is adapted to be received and secured by a housing member 106, and a distal end adapted to be secured to a vertebra. In one embodiment, each distal end is secured to a vertebra using one or more pedicle screw 110 mechanisms. The dimensions of the extension spinal rods can vary, and may be chosen according to factors such as, but not limited to, the existing fixation system, the patient, and the particular surgical application.

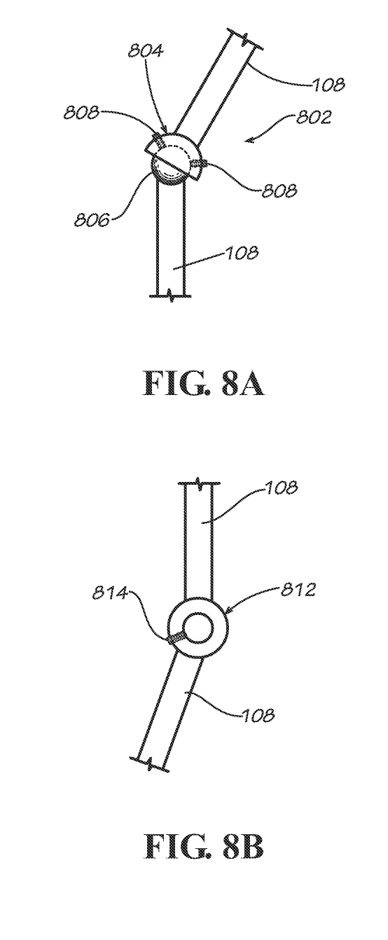

The extension spinal rods 108 may be adjustable in multiple directions, so as to allow proper orientation of the rod 108 to contact the adjacent vertebra despite variations in the location of its proximal end within the housing member 106. According to various embodiments, the adjustment may be achieved by any mechanism known. For example, according to one embodiment, as illustrated in FIG. 8A, an extension spinal rod 108 can include at least one ball joint 802, or other pivot mechanism, at some point along its length, which allows the distal end and/or the proximal end of the extension spinal rod 108 to be moved along one or more of the anteroposterior axis, the superior-inferior axis, and the mediolateral axis. The extension spinal rod 108 may be formed from two members with adjacent ends forming the ball joint 802, one member having a socket end 804, and one member having a ball end 806 configured for fitment within the socket end 804. In one embodiment, the ball joint 802 may include one or more locking mechanisms to substantially lock the ball joint 802 upon reaching the desired orientation. Locking mechanisms may include, but are not limited to, screwing mechanisms 808 (e.g., set screws), which are illustrated by FIG. 8A, or clamping mechanisms, locking pins, a deformable socket end 804, and the like. FIG. 8B illustrates another example embodiment, in which an extension spinal rod 108 includes a planar joint 812 mechanism and screwing mechanism 808 (e.g., a set screw), enabling the extension spinal rod 108 to be moved along a single plane. In one embodiment, the joint 812 and/or the extension spinal rod 108 can be rotated to change the direction of the adjustment.

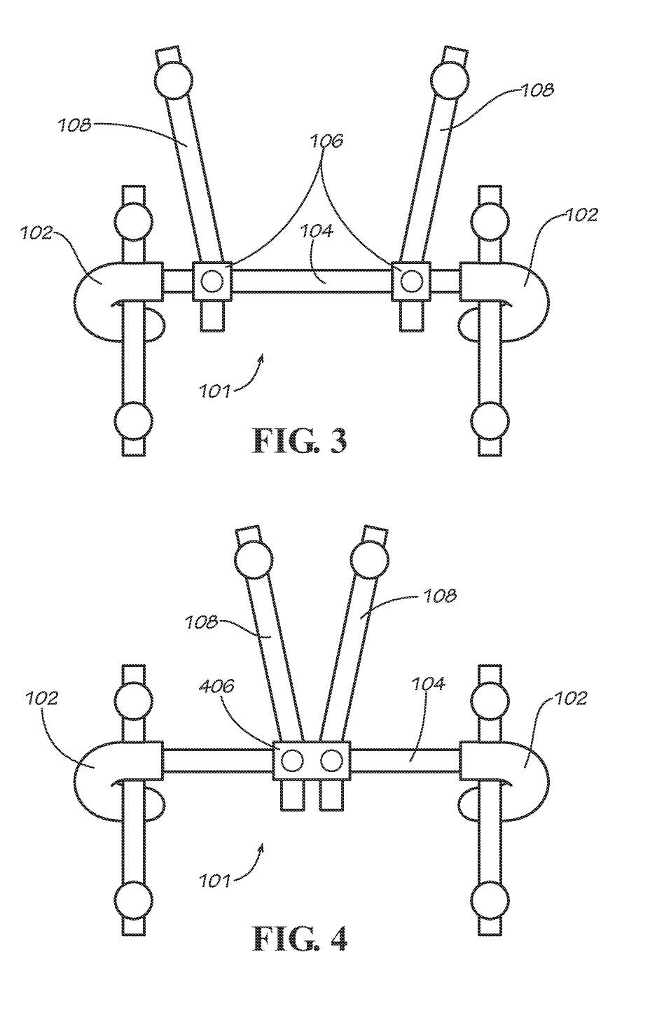

In yet other embodiments, all or a portion of an extension spinal rod 108 can be bendable or otherwise moldable to allow adjustment along one or more of these axes. For example, the extension spinal rods 108 shown in FIG. 2 each have a bend. The extension spinal rods 108 may be designed to have a bend initially, or they may be designed to be straight initially, as shown in FIGS. 3 and 4. Also, depending on the adjustment mechanism, example extension spinal rods 108 may be bent or further adjusted during the surgical procedure. Any of the various example extension spinal rods described with reference to FIGS. 1-4 and 7-8 may also be configured to include a sliding adjustment mechanism, such as the sliding mechanisms discussed above in the context of the connecting member of the transverse connector.

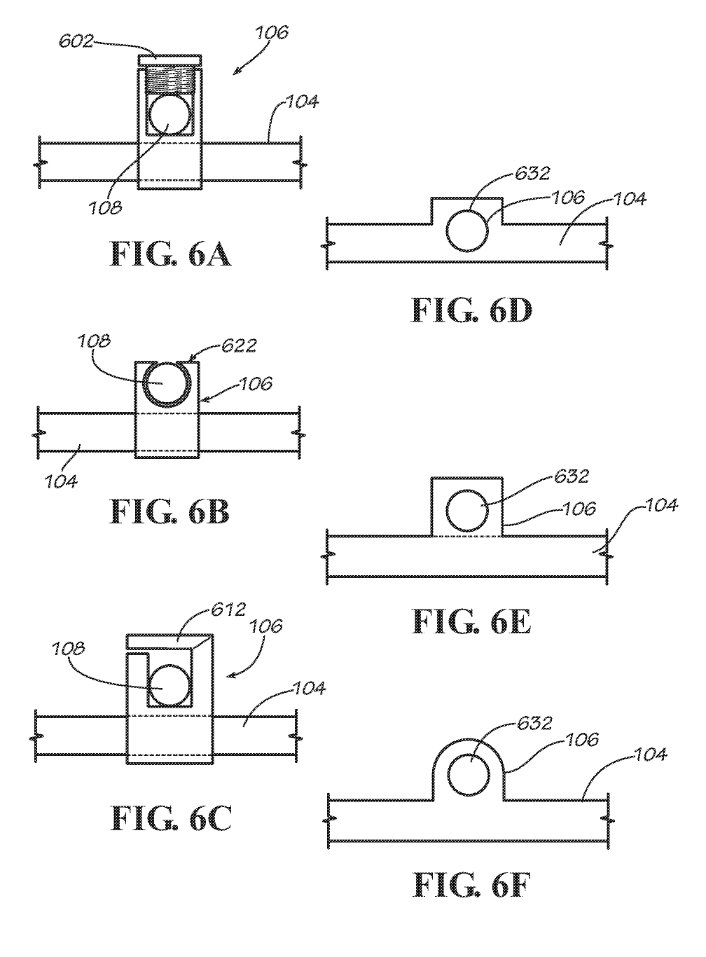

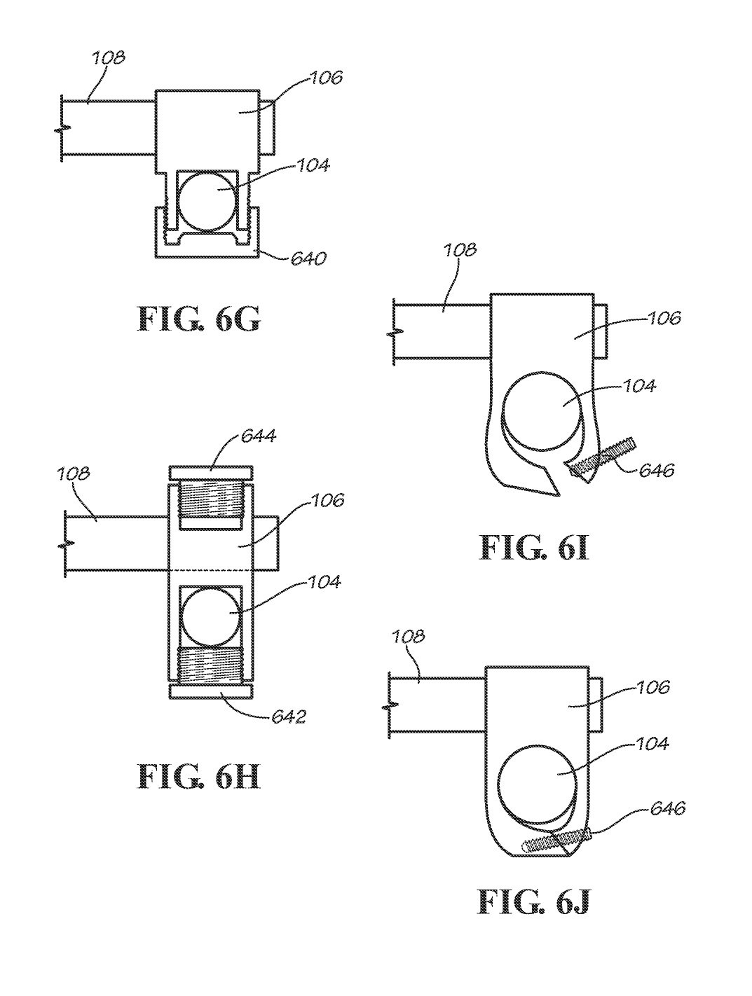

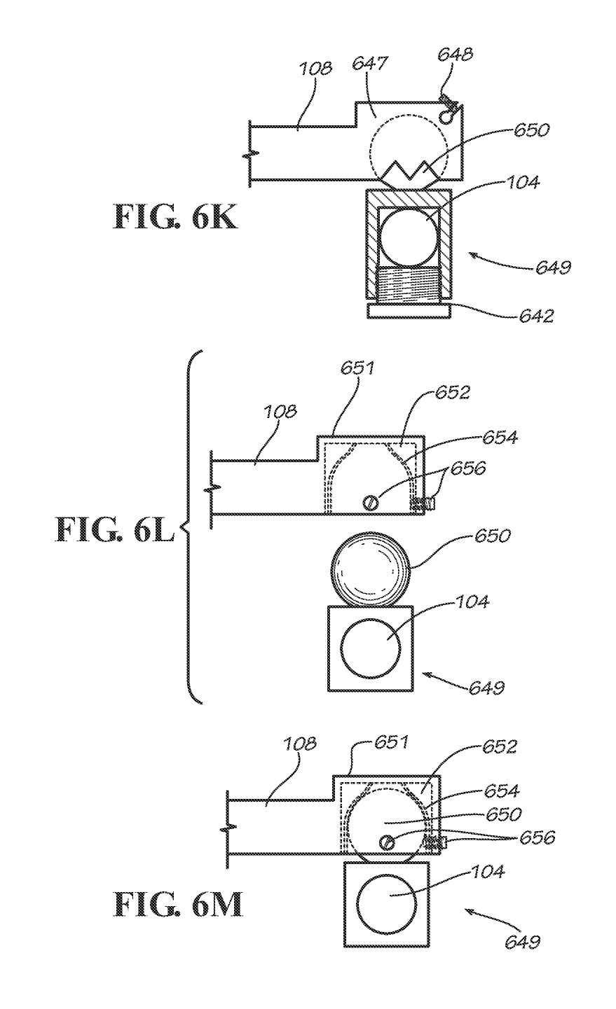

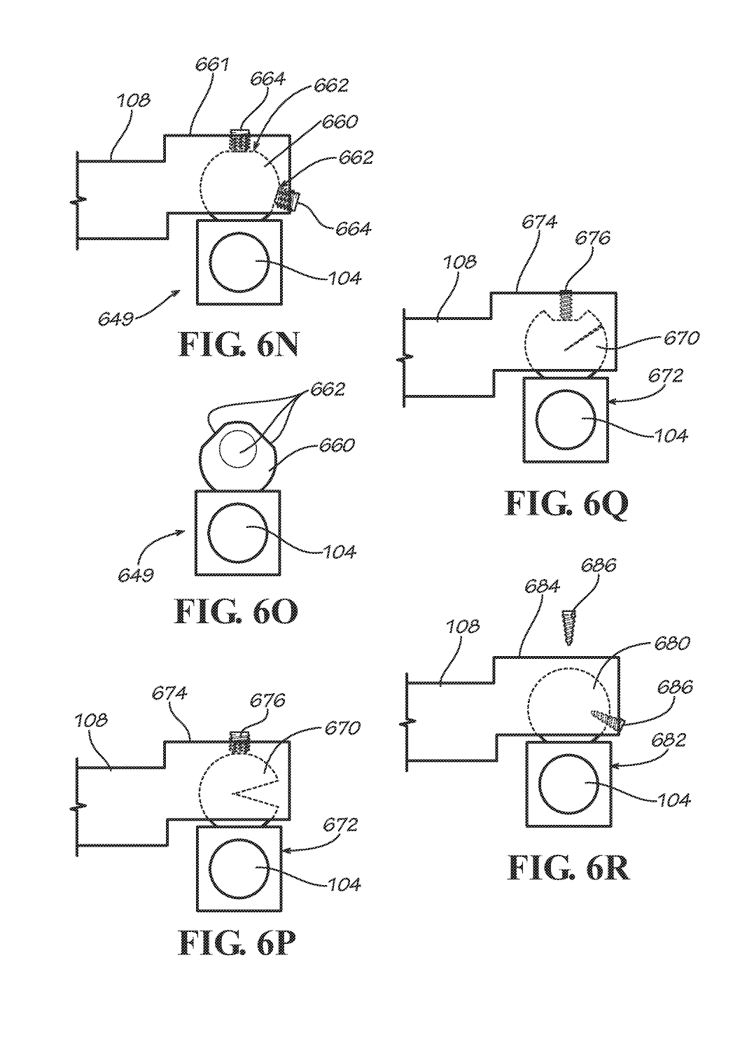

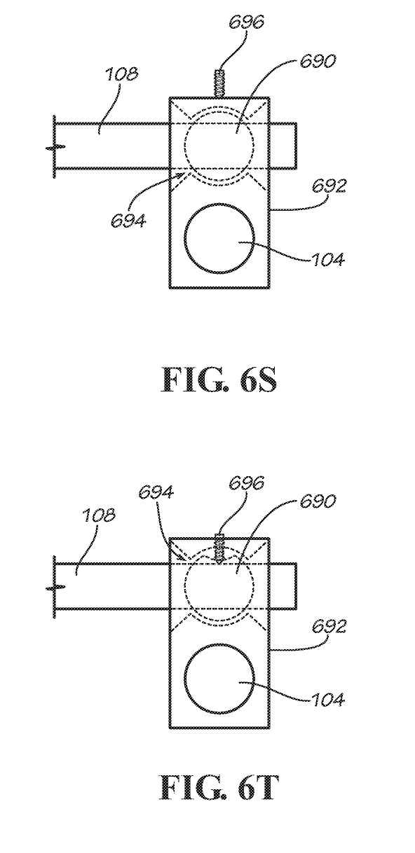

The extension rod device can have one or more housing members for connecting each extension spinal rod 108 to the transverse connector 101. The housing members' configuration and location can vary, as further described with reference to FIGS. 6A-6T. With reference to the entire extension rod device, in some embodiments, there may be two housing members for separately connecting the extension spinal rods 108 to a transverse connector 101, such as the housing members 106 illustrated with reference to FIGS. 1-3. In these embodiments, the housing members 106 can be located close to the lateral ends of the transverse connector 101, or at any other point along the transverse connector 101. In other embodiments, such as is shown in FIG. 4, the extension rod device can include a single housing member 406 configured to receive and secure multiple extension spinal rods 108 within the housing member 406. A single housing member 406 can be configured to include any of the mechanisms shown in and described with reference to FIGS. 6A-6T. It is appreciated that the configuration and the location of the housing member(s), as well as whether they initially connect to an extension spinal rod or to a transverse element, may affect the design, orientation, and procedure for implanting the extension rod device.

As compared to the embodiment illustrated in and described with reference to FIG. 1, the extension rod device embodiments of FIGS. 7A and 7B show extension spinal rods 704, 712 that can be connected to a previously implanted transverse connector 702 disposed between two spinal rods 220 secured by pedicle screws 222. In the embodiment of FIG. 7A, each extension spinal rod 704 includes a housing member 706 at its proximal end and a pedicle screw 708 at its distal end. The housing member 706 can be any housing member illustrated in and described with reference to FIGS. 6A-6T that will secure the extension spinal rods 704 to the transverse connector 702. It is appreciated that in other embodiments, the housing members 706 may be separate components or may be affixed to the transverse connector 702.

In the embodiment of FIG. 7B, two separate extension spinal rods 712 (bent as compared to straight) can include connecting ends 714 for connecting to the transverse connector 702 via a single, separate housing member 710. The separate housing member 710 can be first connected to the transverse connector 702 and subsequently connected to the extension spinal rods 712, or first connected to the extension spinal rods 712 and then to the transverse connector 702. The separate housing member 710 and the connecting ends 714 are illustrated as a ball and socket configuration, such as those shown in and described with reference to FIGS. 6K-6R; however, any other housing member configuration described herein can be used as a separate housing member 710. Moreover, any extension spinal rod configuration can be included in this (or any other) embodiment, such as, but not limited to, those that are bendable, jointed, straight, or bent.

FIG. 6 shows a series of housing member embodiments. FIG. 6A shows a screw cap design, according to one embodiment. With the screw cap 602 removed from the housing member 106, the proximal end of the extension spinal rod 108 is placed in the housing member 106, and the extension spinal rod 108 is secured in place using the screw cap 602. According to one embodiment, the housing member 106 can be configured to include outward facing threads on its upper peripheral edge, and the screw cap 602 can be formed to include complementary threads on its inner surface, permitting the screw cap 602 to be secured to the housing member 106. In one embodiment, the extension spinal rod 108 can have a tight fit within the housing member 106, such that when the screw cap 602 is in place it will secure against the extension spinal rod 108. In another embodiment, the screw cap 602 can further include an extending member extending from its interior, such that when the screw cap 602 is screwed in place on the housing member 106 the extending member will abut and place additional pressure on the extension spinal rod 108. Examples of a screw cap including an extending member are further illustrated in and described with reference to FIGS. 6G and 6H. Although the extension spinal rod is shown to be round, it can have any shape, and the housing member 106 and the screw cap 602 can be configured in complementary shapes to permit a tight fit between the components.

FIG. 6B shows a flip cap design, according to one embodiment. With the flip cap 612 in the open position, the proximal end of an extension spinal rod 108 is placed in the housing member 106, and the extension spinal rod 108 is secured in place using the flip cap 612. The flip cap 612 and/or the housing member 106 may include securing mechanisms for securing the flip cap 612 to the housing member 106, such as, but not limited to, a clamp mechanism, a screw mechanism, and the like. Again, the housing member 106 can be configured so the extension spinal rod 108 has a tight fit within the housing member 106 and the flip cap 612. In addition, according to one embodiment, the flip cap 612 may also include an extending member that can abut and place additional pressure on the extension spinal rod 108, similar to that described with reference to FIG. 6A.

FIG. 6C shows a pressure fit design, according to one embodiment. According to this embodiment, the proximal end of an extension spinal rod 108 can be snapped into a corresponding opening in the housing member 106 and thereby secured. According to this embodiment, the opening in the housing member 106 can be formed to have substantially the same shape as the cross-section of the extension spinal rod 108 that it is designed to secure. Moreover, according to various embodiments, the housing member 106 can be constructed from materials exhibiting slightly elastic properties, such as, but not limited to, metals, metal alloys, polymers, and the like, such that when securing an extension spinal rod 108 into the opening, the opening will deform enough to permit forcing the extension spinal rod 108 in place. Once in place, the housing member 106 can return to its original shape, securing the extension spinal rod 108 therein. It is appreciated that in this embodiment (or any of the others described herein), an additional securing mechanism, such as a securing bolt, a set screw, an adhesive, a weld, and the like, may additionally be employed to secure the fitment and orientation of the extension spinal rod 108 within the housing member 106.

As shown in FIGS. 6A-6C, any of the housing members described herein can be a separate component removably attached to the connecting element 104 of a transverse connector (e.g., the transverse connector 101, 702 of FIGS. 1-4 or 7). In other embodiments, however, a housing member 106 can be a permanent or semi-permanent component of the connecting element 104 of a transverse connector. For example, a connecting element 104 can be molded or otherwise manufactured to include a housing member 106. As one example, FIGS. 6D-6F show sliding designs, in which an extension spinal rod 108 can be slidably inserted through an aperture 632 in a housing member 106 formed as part of a connecting element 104. In the example embodiment of FIG. 6D, an aperture 632 is oriented to extend at least partially through a connecting element 104. Whereas, the example embodiment of FIG. 6E shows a housing member 106 with an aperture 632 positioned above the connecting element 104. Any of the example embodiments shown and described herein, as well as any other housing member embodiment, can be modified to have rounded or substantially smoothed edges, such as is illustrated by FIG. 6F. Also, any of the housing members can be modified to have a substantially minimized profile with respect to the transverse connector, spinal rods, extension spinal rods, and any other implant component. Such design modifications can reduce the bulk of the hardware involved and minimize irritation in the patient's body.

FIGS. 6G-6J show additional embodiments of housing members 106 that can be attached to an extension spinal rod 108 and subsequently connected to a connecting element 104 of a transverse connector, in contrast to those embodiments of FIGS. 6A-6F, which are first attached to a connecting element 104 of a transverse connector. In the embodiment illustrated in FIG. 6G, a housing member 106 can be slidably attached to an extension spinal rod 108. For example, the housing member 106 can include an aperture through which the end of an extension spinal rod 108 can be inserted. In this embodiment, the housing member may be permanently, semi-permanently, or removably affixed to an extension spinal rod 108, such that it may be in place before performing the procedure, or secured to the extension spinal rod 108 and/or adjusted while performing the procedure. The housing member 106 according to this embodiment may further include an opening and a securing mechanism, such as a screw cap 640 described with reference to FIG. 6A, for securing the housing member 106 to a connecting element 104 of a transverse connector. As can be seen by FIG. 6G, the screw cap 640 may include an extending member that will abut the surface of the connecting element 104 when the screw cap is tightened in place. This embodiment further illustrates the outward facing threads of the housing member 106 and the internal threads of the screw cap 640. It is further appreciated that any additional securing mechanisms, such as a securing bolt, a set screw, an adhesive, a weld, and the like, may additionally be employed to secure the fitment and orientation of the extension spinal rod 108 within the housing member 106.

According to another embodiment, as illustrated by FIG. 6H, a housing member 106 may be configured as a separate component, which may first be optionally attached to an extension spinal rod 108 or a connecting element 104 of a transverse connector. To achieve this flexibility, the housing member 106 can include two separate securing mechanisms, one for attaching to an extension spinal rod 108 and one for attaching to a connecting element 104. In the embodiment illustrated in FIG. 6H, the securing mechanisms employ a version of the screw cap design further described herein For example, the housing member 106 can include an extension rod screw cap 644 for threadably securing an extension spinal rod 108 into the housing member 106, and a transverse connector screw cap 642 positioned opposite the extension rod screw cap 644. The screw caps 642, 644 of this embodiment are configured differently than those of FIGS. 6A and 6G, such that the housing member 106 includes internal threads and the screw caps 642, 644 include complementary outward facing threads. In essence, the screw cap of this embodiment resembles a bolt or screw. The housing member 106 of this embodiment can be manufactured by cross-drilling a block with two openings or apertures transversely oriented, and threading opposite ends for receiving the screw caps 642, 644. It is further appreciated that a separate housing member 106, like that of FIG. 6H, can be configured to include any of the other securing mechanisms illustrated and described herein, or any other securing mechanism.

FIGS. 6I and 6J illustrate another embodiment of a housing member 106 that includes an opening forming a deformable sleeve for receiving and deforming around a connecting element 104 of a transverse connector. For example, the housing member 106 can include an opening for inserting the connecting element 104 therethrough (in a manner similar to that describing the snap-fit housing member of FIG. 6C). Upon inserting the connecting element 104 into the opening, one or more screws 646 can be inserted through threads drilled through both open ends forming the opening. Upon tightening the screw 646, the housing member 106 will collapse to close the gap of the open ends, securing the connecting element 104 within the opening, as illustrated by FIG. 6J. It is also appreciated that housing members 106 of this embodiment can be constructed from materials having slightly malleable and/or elastic properties, such as a metal or metal alloy softer than the screw 646 and/or the connecting element 104. It is appreciated that, according to other embodiments, any other means for deforming the housing member 106 around the connecting element 104 may be used, such as, but not limited to, a clamping mechanism, a threaded mechanism, and the like. For example, in another embodiment, a threaded sleeve can thread over threads having an increasing radius formed in the exterior surface of the housing member 106 around the open ends. When the threaded sleeve is tightened over the increasing radius threads, the housing member 106 will deform and collapse onto the connecting element 104, securing it in place.

FIGS. 6K-6O illustrate various embodiments of ball and socket connectors. With reference to FIG. 6K, an extension spinal rod 108 can include a socket end 647 having a substantially hemispherical cavity formed therein to receive a complementary shaped and sized ball 650 on a ball connector 649. The socket end 641 can be configured to include at least one gap and screw 648 opposite the open end. A threaded orifice can be formed through one of the two ends of the socket end 641 forming the gap. When the screw 648 is threadably inserted into the threaded orifice, it extends into the gap and exerts pressure against the surface opposite the threaded orifice. As the screw 648 is tightened against the surface, it causes the size of the gap to increase, which in turn causes the opposite end of the socket 647 to deform and tighten around the ball 650 inserted therein. In the embodiment illustrated, the socket end 647 can include one or more reliefs cut into the edge forming the cavity to facilitate deformation of the socket end 647 around the ball 650 when inserted therein. It is appreciated that the socket end 647 of this embodiment can be constructed from materials having slightly malleable and/or elastic properties, such as a metal or metal alloy softer than the screw 648 and/or the ball 650.

With continued reference to FIG. 6K, the ball connector 649 is illustrated as a separate connector that can be removably attached to a connecting element 104 of a transverse connector using any securing mechanisms, such as a screw cap 642, as described above. The ball connector 649 is configured to include a ball 650 in a substantially spherical shape extending therefrom, which is inserted into the socket end 647 for securing the extension spinal rod 108 to the ball connector 649, and thus to the transverse connector. Although the ball connector 649 is shown as a separate connector, in other embodiments it can be more permanently affixed to and/or integrated with a transverse connector, as described herein.

The embodiment illustrated in FIGS. 6L and 6M, shows a socket end 651 having a slightly different configuration, with FIG. 6L showing the embodiment prior to insertion of the ball and FIG. 6M showing the embodiment after securing the device. The socket end 651 can be configured to include cavity 652 formed within the socket end 651 and a deformable sleeve 654 attached to a portion of the inside surface forming the cavity 652. The deformable sleeve 654 may be substantially hemispherical, sized and shaped to receive a complementary ball 650 of a ball connector 649, as described above. According to this embodiment, after the ball 650 is inserted into the deformable sleeve 654 of the socket end 651, one or more deforming mechanisms 656 (shown as a screw here) can exert force at one or more points on the distal end of the deformable sleeve 654 causing it to crush around the surface of the ball 650, securing it within the socket end 651, as illustrated in FIG. 6M. While a screw is illustrated in FIGS. 6L and 6M, it is appreciated that any other mechanism for exerting a force at one or more points on the deformable sleeve 654 can be used, such as, but not limited to, a clamping 654 of this embodiment can be constructed from materials having slightly malleable and/or elastic properties, such as a metal or metal alloy softer than the screw 656 and/or the ball 650.

FIGS. 6N and 6O illustrate yet another embodiment of a socket end 661 and a ball connector 649. In this embodiment, the ball 660 of the ball connector 649 can be configured as a substantially spherical shape with one or more flattened surfaces 662 formed thereon. The flattened surfaces 662 can be formed in multiple locations around the surface of the ball 660 for receiving one or more set screws 664 to improve the purchase by the set screw 664 on the ball 660. The socket end 661 thus is configured to include a cavity for receiving the ball 660 and one or more threaded orifices formed therethrough to receive one or more set screws 664 for securing the ball connector 649 within the socket end 661, thus securing the extension spinal rod 108 to a connecting element 104 of a transverse connector.

FIGS. 6P and 6Q illustrate yet another embodiment of a socket end 674 and a ball connector 672. In this embodiment, the ball 670 of the ball connector 672 can also be configured as a substantially spherical shape, but including one or more detents formed into the ball 670. The detents permit the ball 670 to crush or otherwise deform when a screw 676 (or multiple screws) are tightened through the socket end 674 into the surface of the ball 670, as shown in FIG. 6Q. By deforming the ball 670 within the socket end 674, the extension spinal rod 108 can be secured in an exact orientation, further restricting its movement around the ball 670. The ball 670 can be constructed from materials having slightly malleable and/or softer properties, such as a metal, a metal alloy, a polymer, or any combination thereof, softer than the screw 676. The ball 670 may be substantially solid or at least partially hollow, according to various embodiments. For example, an at least partially hollow ball 670 may improve the ease with which the ball 670 can be deformed by the screw or screws 676, while also increasing the additional volume occupied by the deformed ball 670 within the socket end 674. Moreover, although the detent is illustrated as being wedge shaped in FIG. 6P, detents may be formed in any other shape.

FIG. 6R illustrates yet another embodiment of a socket end 684 and a ball connector 682. In this embodiment, the ball 680 of the ball connector 682 can be configured as a substantially spherical shape and constructed at least partially from a polymer, such that sharpened screws 686 can be inserted through the socket end 684 and into the polymer surface of the ball 680. In one embodiment, the ball 680 may be formed completely or substantially from a polymer or a combination of polymers. Though, in other embodiments, the ball 680 may include a metallic core with a pliable coating having a thickness great enough to receive the end of a sharpened screw or screws 686. The pliable coating may be constricted from a polymer, a metal, a metal alloy, or any combination thereof, that may adequately receive and retain the screw 686. It is appreciated that the thickness of the pliable coating may very, depending upon the size and application of the extension rod device, but may range anywhere from less than 1 mm to at least 10 mm thick; though, in other embodiments, the pliable coating may be greater than 10 mm thick.

FIGS. 6S-T illustrate yet another embodiment of a housing member that includes a ball and socket mechanism. According to this embodiment, the housing member 692 can include an aperture (or any other securing mechanism shown or described herein) for attaching to a connecting element 104 of a transverse connector. The housing member 692 further includes a socket opening 694 formed on the opposite end. The socket opening 694 can be formed within the housing member 692 to have a substantially hollow spherical shape therein for receiving a ball 690 and at least one opening through which an extension spinal rod 108 may be inserted. In the embodiment illustrated in FIGS. 6S-T, the extension spinal rod 108 is shown as extending entirely through the socket opening 694; though, in other embodiments, the socket opening 694 may have only a single opening to receive an extension spinal rod 108 on one end. The ball 690 is configured as a loose ball that complements the shape and size of the socket opening 694 and rotatably fits therein. The ball 690 further includes an aperture formed therethrough configured to receive an extension spinal rod 108.

Accordingly, once the extension spinal rod 108 is inserted through the ball 690 contained within the housing member 692, one or more screws 696 can be tightened through the housing member 692, into the socket opening 694, and exert a force at one or more positions on the ball 690. In one embodiment, as shown in FIG. 6T, the ball 690 is a deformable ball such that the force exerted by the one or more screws 696 causes the ball 690 to deform (e.g., displace radially from the point of pressure by the screw(s) 696) around the extension spinal rod 108 and further fill or wedge within the socket opening 694. The ball 690 can be deformable at least in part due to the material from which it is constructed, such as being constructed from materials having slightly malleable and/or softer properties, such as a metal, a metal alloy, a polymer, or any combination thereof, softer than the screw 696 and the socket opening 694. In other embodiments, the ball 690 can also, or instead, be deformable due at least in part to its shape, such that one or more detents, slots, or other voids are formed into the surface of the ball 690 to facilitate deformation under the pressure of the screw(s) 696.

In one embodiment, to ease the manufacture and assembly of the ball 690 and the housing member 692, the ball 690 can be formed as partially spherical, with the aperture for receiving the extension spinal rod 108 forming flattened ends on opposing sides, which causes the ball 690 to have a greater diameter perpendicular to the aperture than the diameter through the aperture. Thus, this smaller diameter may be dimensioned so as to permit the ball 690 to be inserted into the socket opening 694 when rotated such that the smaller diameter fits into the socket opening 694, but when rotated to expose the aperture to the opening(s), the ball 690 will be retained within the socket opening 694. In other embodiments, the ball 690 and housing member 692 may be manufactured together, and delivered with the ball 690 installed.

Any of these housing member embodiments or other connecting mechanisms illustrated as being first connected to or integrated with a connecting element of a transverse connector can instead be first connected to or integrated with an extension spinal rod, and any embodiments illustrated as being first connected to or integrated with an extension spinal rod can instead be first connected to or integrated with a connecting element of a transverse connector. An extension spinal rod may be inserted into and secured in a housing member prior to the surgical procedure. This could be performed by a physician prior to the procedure, or the device could be packaged and sold with the extension spinal rods pre-secured. Alternatively, the extension spinal rods may be inserted into the housing members during the surgical procedure, such as may be performed if connecting extension spinal rods to an existing implanted transverse connector, or when connecting extension spinal rods to a new transverse connector before or after the lateral ends of the transverse connector have been secured to the existing fixation system.

Any of the extension rod device embodiments illustrated and/or described herein can further include a shielding frame or cap positioned over any or all of the screws or other securing mechanisms to prevent the screws or other securing mechanisms from backing out. A shielding frame or cap can further shield and protect adjacent tissue structure from irritation, erosion, or other injury from partially extending screws, etc. The shielding frame(s) or cap(s) may be pressure fit or snap fit over any or all of the screws or other mechanisms (e.g., pedicle screws, housing assemblies, set screws used with the housing assemblies, etc.), or may be secured by any other means, as is known.

Moreover, one or more components of the various embodiments of the extension rod device described herein can be manufactured, packaged, and provided as a kit. An extension rod device kit may include any or all of: one or more extension rods, one or more pedicle screws, one or more housing members and associated securing components, one or more transverse connectors, assembly tools, and/or removal tools. For example, one extension rod device kit may include only extension rods and pedicle screws and corresponding securing mechanisms, such as if the kit is to be attached to an existing transverse connector with existing housing members. In another example, an extension rod device kit may include extension rods, pedicle screws, and housing members and associated securing components, which can be used to attach to an existing transverse connector that does not incorporate housing members. Yet another extension rod device kit may include extension rods, pedicle screws, a transverse connector, and the housing members with associated securing components, for implantation in a system that only has existing spinal rods in place but no transverse connector or when replacing an existing transverse connector.

The extension rod device and its component parts may be made of any biocompatible material. For example, the extension rod device may be constructed from entirely or partially metallic materials, such as, but not limited to, nickel, titanium, stainless steel, tantalum, titanium, gold, cobalt chromium alloy, or any combination thereof. In another example, the extension rod device may be constructed from polymeric material, such as but not limited to, epoxy, polypropylene, polyethylene, polyamide, polyxylene, polyvinyl chloride (“PVC”), polyurethane, polyetheretherketone (“PEEK”), polyethylene terephthalate (“PET”), liquid crystal polymer (“LCP”), or any combination thereof. In some embodiments, the extension rod device may be constructed from a combination of one or more of these polymeric or metallic materials. Other biocompatible materials known in the art to be suitable for fabricating or encasing implantable medical devices also may be used.

Spinal fixation devices are typically implanted in the lumbar region, although they can be implanted in other regions of the spine. The extension rod device embodiments disclosed herein can be adapted to extend an existing fixation system in any region of the spine. Also, the extension rod device can be implanted in an open or minimally invasive procedure using known surgical techniques. If a transverse connector is already in place on the existing fixation system, embodiments of the extension rod device can be secured to the existing transverse connector, or the existing connector could be removed to make room for an extension rod device that includes a new transverse connector.

Many modifications and other embodiments of the exemplary descriptions set forth herein to which these descriptions pertain will come to mind having the benefit of the teachings presented in the foregoing descriptions and the associated drawings. Thus, it will be appreciated that the invention may be embodied in many forms and should not be limited to the exemplary embodiments described above. Therefore, it is to be understood that the invention is not to be limited to the specific embodiments disclosed and that the modifications and other embodiments are intended to be included within the scope of the appended claims. Although specific terms are employed herein, they are used in a generic and descriptive sense only and not for purposes of limitation.

What is claimed is:

1. A transverse connector device for extending a spinal fixation device, comprising:
a first lateral end adapted to engage a first spinal fixation element;
a second lateral end adapted to engage a second spinal fixation element;
a connecting element therebetween; and
at least one housing member adapted to receive and secure at least one extension spinal rod.

2. The transverse connector device of claim 1, wherein the first second spinal fixation and the second spinal fixation element are spinal rods.

3. The transverse connector device of claim 1, wherein the connecting element is adjustable along the superior-inferior axis.

4. The transverse connector device of claim 1, wherein the connecting element is adjustable along the anteroposterior axis.

5. The transverse connector device of claim 1, wherein the connecting element is adjustable in length.

6. The transverse connector device of claim 1, wherein the first lateral end and the second lateral end are secured to the first second spinal fixation and the second spinal fixation element, respectively, using attachment members.

7. The transverse connector device of claim 1, wherein the first lateral end and the second lateral end each comprise a clamp.

8. The transverse connector device of claim 1, wherein the first lateral end and the second lateral end each comprise a hook.

9. The transverse connector device of claim 1, further comprising at least one spinal rod having a proximal end adapted to be received and secured by the housing member and a distal end adapted to be secured to a vertebra.

10. The transverse connector device of claim 9, wherein the distal end of the at least one spinal rod is secured to the vertebra using a pedicle screw.

11. An extension spinal rod for extending an existing spinal fixation system, comprising:
a proximal end adapted to be secured to a transverse connector; and
a distal end adapted to be secured to a vertebra,
wherein the extension spinal rod is configured to extend from a transverse connector extending between spinal fixation elements at a first vertebral level to at least one vertebra at an adjacent vertebral level.

12. The extension spinal rod of claim 11, wherein the extension spinal rod is adjustable along the superior-inferior axis.

13. The extension spinal rod of claim 11, wherein the extension spinal rod is adjustable along the anteroposterior axis.

14. The extension spinal rod of claim 11, wherein the extension spinal rod is adjustable along the mediolateral axis.

15. The extension spinal rod of claim 11, further comprising at least one housing member attachable to the proximal end of the extension spinal rod, wherein the housing member is further attachable to the transverse connector.

16. The extension spinal rod of claim 15, wherein the at least one housing member comprises at least one of: a screw cap; a flip cap; a snap fit aperture; a ball and socket mechanism, a deformable sleeve; a set screw; a deformable socket and ball mechanism; or a deformable ball and socket mechanism.

17. The extension spinal rod of claim 15, wherein the at least one housing member comprises a socket opening and a ball rotatably positioned therein; and wherein the ball comprises an aperture for receiving the proximal end of the at least one extension spinal rod.

18. The extension spinal rod of claim 17, wherein the housing member further comprises at least one screw insertable through the housing member and into the socket opening for exerting a force on the ball, wherein the at least one screw is adapted to exert a force on the ball and deform the ball around the at least one extension rod.

19. A method of extending an existing spinal fixation system to adjacent vertebrae, comprising:
securing a first lateral end of a transverse connector to a first implanted spinal fixation element;
securing a second lateral end of the transverse connector to a second implanted spinal fixation element;
securing a proximal end of at least one extension spinal rod to the transverse connector; and
securing a distal end of the at least one extension spinal rod to a vertebra.

20. A method of extending an existing spinal fixation system, having an existing transverse connector, to adjacent vertebrae, comprising:
securing a proximal end of at least one extension spinal rod to an existing transverse connector; and
securing a distal end of the at least one extension spinal rod to a vertebra.

21. The method of claim 20, wherein securing the proximal end of the at least one extension spinal rod comprises:
attaching at least one housing member to the transverse connector; and
attaching the proximal end of the at least one extension rod to the at least one housing member.

22. The method of claim 21, wherein the at least one housing member comprises a ball connector comprising a ball extending therefrom, and wherein the proximal end of the at least one extension spinal rod comprises a socket end for receiving the ball of the ball connector.

23. The method of claim 22, wherein the socket end further comprises a deformable sleeve, and wherein securing the proximal end of the at least one extension spinal rod further comprises:
placing the deformable sleeve over the ball of the ball connector; and
deforming the deformable sleeve over the ball.

24. The method of claim 22, wherein the ball of the ball connector is at least partially deformable, and wherein securing the proximal end of the at least one extension spinal rod further comprises:
placing the socket end over the ball of the ball connector; and
advancing at least one screw through the socket end and into the ball to deform the ball.

25. The method of claim 22, wherein the ball of the ball connector comprises a pliable coating at least partially covering the ball, and wherein securing the proximal end of the at least one extension spinal rod further comprises:
placing the socket end over the ball of the ball connector; and
advancing at least one screw through the socket end and into the pliable coating.

26. The method of claim 22, wherein the socket end is removably attachable to the proximal end of the at least one extension spinal rod.

27. The method of claim 21, wherein the at least one housing member comprises a socket opening and a ball rotatably positioned therein, and wherein the ball comprises an aperture for receiving the proximal end of the at least one extension spinal rod, and further comprising tightening the at least one screw through the housing member and deforming the ball around the at least one extension spinal rod.

28. An extension rod device kit, comprising:
at least one extension spinal rod having a proximal end and a distal end;
at least one pedicle screw assembly for affixing the distal end of the at least one extension spinal rod to a vertebra; and
at least one housing member for receiving the proximal end of the at least one extension rod and for securing to a transverse connector securable between two implanted spinal rods.

29. The extension rod device kit of claim 28, further comprising the transverse connector.
